(19) United States
(12) Patent Application Publication
(10) Pub. No.: US 2008/0249064 A1
(43) Pub. Date: Oct. 9, 2008

(54) Activities of an active principle obtained from powdered alfalfa seeds (Medicago sativa), active principle used and process of obtaining the same

(75) Inventor: Jean Paufique, Objat (FR)

Correspondence Address: YOUNG & THOMPSON, 209 Madison Street, Suite 500, ALEXANDRIA, VA, 22314, US

(73) Assignee: SOCIETE INDUSTRIELLE LIMOUSINE D'APPLICATION BIOLOGIQUE DITE SILAB, OBJAT (FR)

(21) Appl. No.: 11/902,836

(22) Filed: Sep. 26, 2007

Related U.S. Application Data
(62) Division of application No. 11/047,854, filed on Feb. 2, 2005, now abandoned.

(30) Foreign Application Priority Data
Feb. 2, 2004 (FR) 04 00986

(51) Int. Cl. A61K 31/715; A61P 17/00

(52) U.S. Cl. 514/54

(57) ABSTRACT

The object of the invention is an active principle derived from a powder of alfalfa seeds (Medicago sativa) permitting counteracting, in a manner similar to retinol, the phenomenon of extrinsic aging of the skin, with anti-wrinkle and smoothing properties. The invention also covers the active principle, the procedure of obtaining the same, and the compositions including it.

### BACKGROUND OF THE INVENTION

1. Field of the Invention

The present invention covers an active principle from powdered alfalfa seeds so as to modulate the expression of a large number of epidermal proteins, to act on the cellular renewal of fibroblasts, to maintain good cellular function, to stimulate the synthesis of collagen I by inhibting the action of the enzymes responsible for its degradation.

The invention also covers the active principle used, as such and its process of obtention.

2. Description of the Related Art

Given the duration of life, which increases, the appearance should also be increased, particularly of the face by which often judgment on one is passed by others, namely by correlation.

The cosmetic industry seeks to counter cutaneous aging, which remains a complex process which affects all the layers of skin.

Studies have led to attributing important proportions to the phenomenon of extrinsic aging, about 75%, with respect to so-called intrinsic aging connected to personal genetics, which is only 25%.

The most important factors are external attacks, sun, pollution, food, poor hygiene of life in general, hence essentially the free radicals if one considers the molecular scale.

More generally, the signs are manifested by a decrease in the barrier function of the skin and cutaneous slackening, particularly arising from loss of the mechanical properties of the skin. The fibroblasts have a slowed metabolic activity. The elastin fibers degrade. The synthesis of collagen, and hence its content, decreases in the skin. The content of proteoglycans decreases and there results a loss of viscoelastic properties of the derma.

So as to counter these phenomena, there are known the activities of retinol or vitamin A and its derivatives on the cells of the skin. The retinoids play an essential role in the embryogenesis and control of cellular growth and their transport by the topical route is used for the treatment of certain maladies of the skin.

This use by the topical route improves the characteristics of aging skin, acting on wrinkles, timelines and/or even the color of the skin.

Nevertheless, in the family of retinoids, there have been noted secondary effects arising from the use of retinoic acid. Particularly, this acid is strongly irritating, preventing its cosmetic application.

As to retinol, it is highly unstable in light, in oxygen, and in heat, and above all very irritating. Retinaldehyde or its ester forms is more stable but penetrates the skin less easily.

### BRIEF SUMMARY OF THE INVENTION

The object of the present invention is to provide an active principle which has effects of the type of those of retinol without having the drawbacks and secondary effects. The invention also covers the process of obtaining it and the compositions including it.

### BRIEF DESCRIPTION OF THE DRAWINGS

FIG. 1 shows the elution curve obtained from gel filtration chromatography of the glucidic fraction of the active principle obtained from alfalfa seeds (Medicago sativa).

FIG. 2 shows the synthesis of collagen I for fibroblasts incubated in the presence or not of the active principle, of the fractions or of retinol.

FIG. 3 shows the effects of the complete active principle, the fraction F1, and retinol on synthesis of cytokeratin-4 (CK4).

FIG. 4 shows the results obtained from gas phase chromatography analysis of the active fraction F1.

FIG. 5 shows the effects of the active principle dosed at 2% and 3%, on the synthesis of the cytokeratin 4 (CK4) compared with those arising from retinol.

FIG. 6 shows the effects of the active principle dosed at 2% and 3%, on the synthesis of the cytokeratin 9 (CK9) compared with those arising from retinol.

FIG. 7 show the results of a study carried out on human keratinocytes treated with retinol as a basis of comparison and with the active principle dose at 0.5%, 1% and 2%.

FIG. 8 compares the expression of RNAm of profilaggrin from human keratinocytes that are incubated in the presence of the active principle at 0.5%, 1% and 2% and retinol at 10⁻⁵ M.

FIG. 9 compares the percentage of cellular viability of human fibroblasts incubated with the active principle and retinol in either a medium deficient in calcium added with growth factors or a medium deficient in calcium and not with added growth factors.

FIG. 10 compares the synthesis of collagen I for fibroblasts treated with retinol at 10⁻⁵ M and with the active principle at 0.1%, 0.25% and 0.5%.

FIG. 11 compares the effect on anti-MMP-1 activity for fibroblasts treated with retinol at 10⁻⁵ M or the active principle at 0.25%, 0.5% and 1%, and untreated.

FIG. 12 compares two compositions: the active principle at 4% in an emulsion and retinol at 0.15% in an emulsion.

FIG. 13 compares the anti-wrinkle effects for the two compositions of FIG. 12.

FIG. 14 shows two compositions used for the evaluation of a smoothing effect: the active principle at 4% in an emulsion and retinol at 0.15% in an emulsion.

### DETAILED DESCRIPTION OF THE INVENTION

As in the case of retinoids, the active principle according to the invention seeks to increase the barrier effect of the stratum corneum and to modulate the expression of a large number of epidermal proteins.

Thus, the active principal acts on cellular renewal of the fibroblasts and takes part in maintaining the functioning of the extracellular matrix.

The active principle stimulates the synthesis of collagen I whilst inhibiting the action of the enzymes responsible for its degradation.

More particularly, the active principle influences in a positive way the keratinocytic differentiation by known different markers such as:

cytokeratin 4,

cytokeratin 19,

HSP 27, and

profilaggrin.

The process according to the invention consists in using the seeds of alfalfa (Medicago sativa) as the natural basis of origin. These seeds contain a high proportion of sugar, oligo-elements and amino acids.

In the process according to the invention:

the powdered alfalfa seeds (Medicago sativa) are dissolved in the amount of at least 50 g/l in an aqueous medium,

this preparation is simultaneously and/or consecutively hydrolyzed in the presence of proteases and carbohydrates,

the soluble and insoluble fractions are separated,

the enzymes are inactivated, and

the active fraction is concentrated by any suitable means such as decantation, filtration, osmosis, inverse osmosis or nanofiltration.

### 1/Active Principle:

There is obtained by this process an active principle which can be characterized by different parameters.

### 1-1/Quantity of Dry Material:

There is used a specimen placed in an oven at 105° C. to obtain constant weight.

The quantity of dry material obtained is 10 to 200 g/l, preferably from 40 to 55 g/l.

### 1-2/Content of Total Sugar:

There is utilized the method of DUBOIS (DUBOIS M. et al. [1956], Analytical Chemistry, 28, No. 3 pages 350-356).

In the presence of concentrated sulfuric acid and phenol, the reductive sugars give a yellow-orange compound.

From a standard range of mannose, glucose, galactose, ranging from 25 μg/l to 100 μg/l, there can be determined the amount of total sugar in a specimen.

The amount of total sugar of the active principle according to the present invention is from 4 to 90 g/l, preferably from 17 to 25 g/l.

### 1-3/Characterization of the Simple Sugars:

To study the simple sugars, there is determined a) the quantity of each of the sugars and b) the degree of polymerization.

a) HPLC analysis gives the following quantity of carbohydrates present in the active principle according to the present invention:

8.4% gluclose,

39.1% mannose, and

52.5% galactose.

b) Analysis of the degree of polymerization shows:

41.7% of monosaccharides with a polymerization degree of 1,

50.1% of disaccharides with a polymerization degree of 2,

4.1% of oligosaccharides with a degree of polymerization of 5, and

4.1% of oligosaccharides and polysaccharides with a degree of polymerization greater than 7.

The glucidic fraction of this active principle obtained from alfalfa seeds (Medicago sativa) is comprised essentially of galactose, glucose and mannose in the form of mono, di- and oligosaccharides.

More particularly, there is found a composition of the glucidic fraction in the form of monosaccharides and disaccharides of at least 90%, and in the form of oligosaccharides and polysaccharides for the rest.

1-4/Determination of the Active Fraction of this Glucidic Composition:

The sugars of this active principle are fractionated by gel filtration chromatography.

The detection is carried out at 280 nm, the elution buffer being a sodium chloride and phosphate buffer.

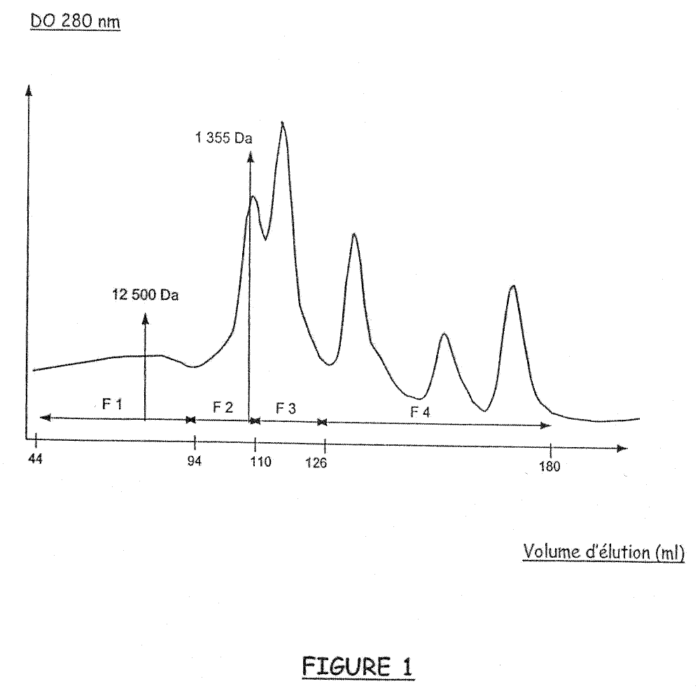

FIG. 1 shows the obtained curve.

The elution volumes of the fractions are as follows:

fraction F0, 0-44 ml

fraction F1, 44-94 ml

fraction F2, 94-110 ml,

fraction F3, 110-126 ml, and

fraction F4, 126-180 ml.

a/Synthesis of Collagen I:

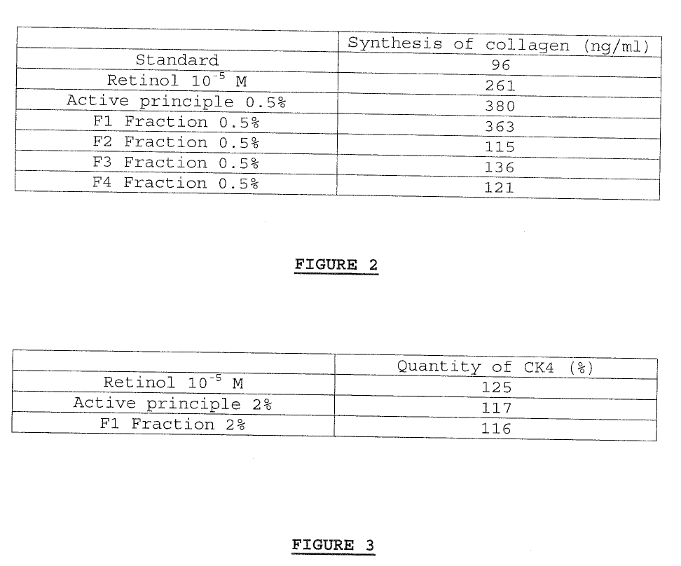

The study is conducted on human fibroblasts by ELISA dosage. The fibroblasts are incubated in the presence or not of the active principle, of the fractions or of retinol at 10⁻⁵ M. The results are shown in FIG. 2.

The fraction F1 is as active as the complete active principle on the synthesis of collagen I, respectively 363 ng/ml and 380 ng/ml.

It is the fraction F1 which confers on the active principle its activity as to the synthesis of collagen I.

b/Synthesis of Cytokeratin-4:

The object is to show that the fraction F1 identified as the active fraction is that also for the keratinocytic differentiation.

The table of FIG. 3 shows that the effect of the complete active principle is due to the fraction F1, and that this effect is comparable to that of retinol. It is also the polysaccharide fraction F1 which confers on the active principle the desired properties.

### 1-5/Analysis of the Active Fraction F1:

Neutral monosaccharides are dosed in the active principle in its raw condition and into the active fraction F1, by the method of DUBOIS mentioned above. The results are as follows:

raw active principle: 20 g/l

active fraction F1: 5.9 g/l

The detail of the composition in neutral monosaccharides is obtained by analysis with the help of the method of Kamerling et al. (1975) modified by Montreuil et al. (1986). The principle rests on the obtention of trimethylsylated methylglucosides by methanolysis followed by pertrimethylsylilation from the monosaccharides constituting the sample.

The analysis has been carried out by gas phase chromatography.

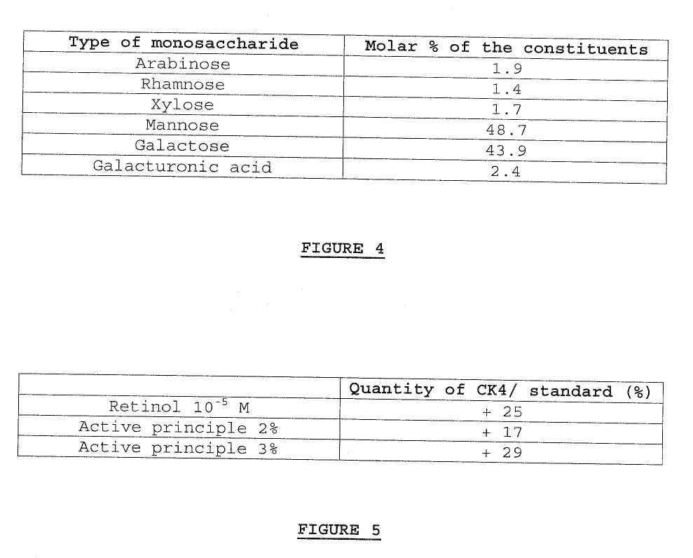

The results obtained are indicated in the table of FIG. 4.

It will be seen that the active fraction F1 is a polysaccharide fraction rich in galactomannanes.

### 2/Identification and Quantification of the Active Principle as to its Formula:

It is sought by this step to identify and quantity the active principle dose with 1% in an emulsion.

The composition of this emulsion is the following:

cetearyl octanoate (Lanol 1688): 10%

arachidyl alcohol/behenyl alcohol/arachidyl glucosid (Montanov 202): 3%

isononyl isononanoate (Lanol 99): 2%

polyacrylamide/isoparaffin C13-14/laureth-7 (Sepigel 305): 2%

active principle: 1%

phenonip: 0.5%, and

water: quantity sufficient for 100%

The emulsionized cream is dephased to recover the aqueous phase which is analyzed by HPLC (high performance liquid chromatography) with the active principle of p-coumaric acid of the utilized marker.

The results give a quantification of peak surface relative to the marker of: 5.77/648.4, namely a concentration of 0.9% of active principle in the composition in emulsion.

The active principle is found to be formulated at 1% in almost its entirety.

### 3/Cosmetic Effectiveness in Vitro:

### 3-1/Keratinocytic Differentiation:

### Synthesis of Cytokeratins 4 and 19:

The study bears on human keratinocytes. A portion is treated with active principle according to the invention and another portion is treated with retinol for comparison.

The total proteins are then dosed by the PCA method (Sigma).

There is carried out an electrophoresis of the cellular extracts brought to the same quantity of proteins on a gel. The proteins are transferred to a membrane.

An immuno-marking is then carried out of the two types of cytokeratin with suitable antibodies.

There are thus seen the effects of the active principle dosed at 2% and 3%, on the synthesis of the cytokeratins 4 and 19 compared with those arising from retinol.

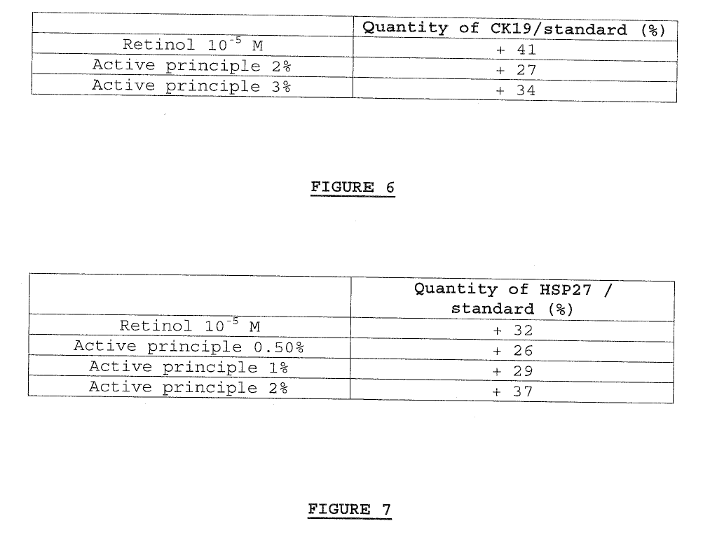

The results are indicated in the tables of FIGS. 5 and 6 respectively. It will be seen that the results are substantially identical to those obtained with retinol, thereby showing that the active principle extracted from alfalfa increases significantly the significance of the cytokeratins 4 and 19 particularly.

### Synthesis of HSP 27 (Heat Shock Protein):

This protein is a marker for the process of keratinocytic differentiation. The study is carried out on human keratinocytes treated with retinol as a basis of comparison and with the active principle dose at 0.5%, 1% and 2%.

There is used the same protocol as before, with suitable immunomarkers and developers.

There are obtained the results shown in FIG. 7.

The active principle according to the present invention permits promoting significantly the synthesis of HSP 27 of 26%, starting from 0.5%.

### Synthesis of Profilaggrin:

Profilaggrin is also implicated in the process of differentiation. Particularly, profilaggrin is transformed into filaggrin, which molecule permits the aggregation of the filaments of cytokeratin.

It is known that retinol stimulates the synthesis of this protein under the conditions of this study.

The protocol which will be followed permits determining the capacities of the active principle from alfalfa to stimulate also the synthesis of this protein.

Human keratinocytes are incubated in the presence of the active principle at 0.5%, 1% and 2%.

The cells are recovered and the total RNA is extracted. After analysis, there is obtained the percentages of the expression of RNAm of profilaggrin relative to this standard.

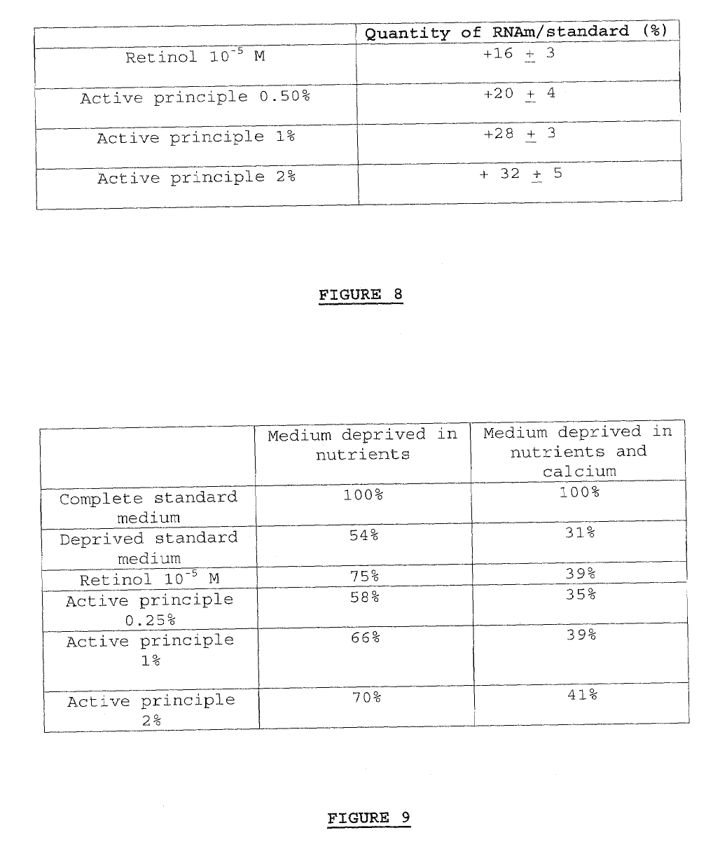

The results are indicated in the table of FIG. 8.

There will be seen a stimulation of the synthesis of profilaggrin of human keratinocytes at least comparable to that of retinol at 10⁻⁵ M.

### 3-2/Influence on the Metabolism of Dermal Cells:

### Effect on the Cellular Proliferation of Human Fibroblasts:

The study consists in comparing the capacity of the active principle to promote the replication and proliferation of human fibroblasts to that of retinol.

Human fibroblasts are incubated either in a medium deficient in calcium added with growth factors, or else in a medium deficient in calcium and not with added growth factors.

There is determined the percentage of cellular viability and the results are given in the table of FIG. 9.

It will be seen that the addition of active principle according to the invention tends to restore a capacity for proliferation of the fibroblasts by compensating the deficiencies of the medium. At 2%, it is seen that the active principle has an activity of stimulation of the cellular proliferation similar to that of retinol.

### Effect on Proteic Metabolism:

The object is to evaluate the effect of the active principle on the synthesis of collagen I, which is a major part of the derma.

Tests have been carried out on normal human fibroblasts by ELISA dosage. The fibroblasts were treated with retinol at 10⁻⁵ M or with the active principle at 0.1%, 0.25% and 0.5%.

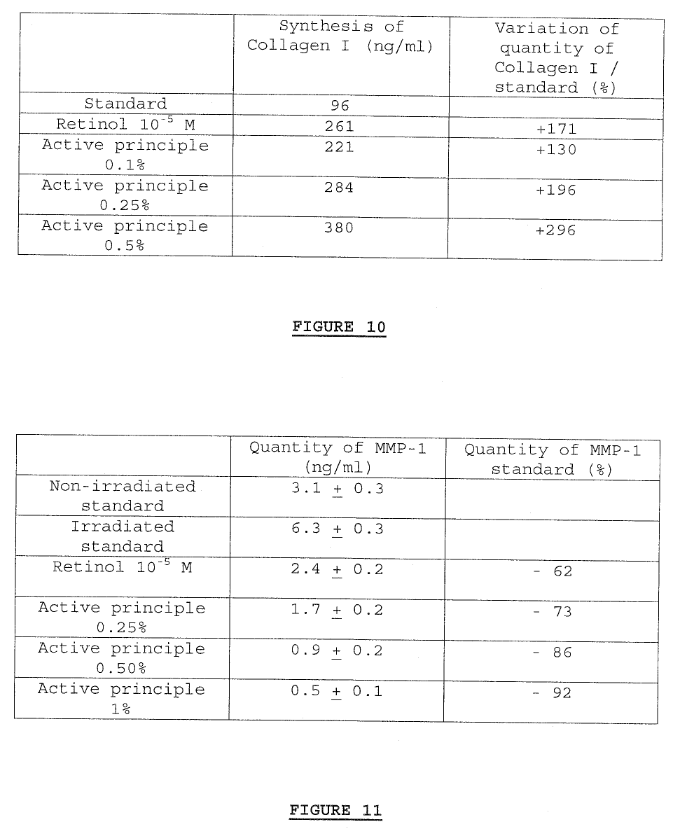

The results are given in the table of FIG. 10, and show that the active principle from alfalfa stimulates significantly the synthesis of collagen I in a dose-dependent way, for example, an increase of 296% for a dosage of 0.5%. It will be noted by comparison that the retinol at 10⁻⁵ M leads to an increase of 171%.

### Effect on the Anti-MMP-1 Activity for the Protection of the Proteins of the Matrix:

The study bears on a culture of human fibroblasts subjected to exposure to ultraviolet light. The quantity of MMP-1 increases to the level of the extra-cellular matrix in the case of aggression of this type.

The fibroblasts are treated with retinol at 10⁻⁵ M, or treated with the active principle at 0.25%, 0.5% and 1% or else untreated.

The results are given in the table of FIG. 11.

It will be seen that the active principle inhibits the synthesis of MMP-1 in a dose-dependent manner. The capacity of the active principle is comparable to that of retinol.

### 3-3/Effect of the Active Principle on the Differential Expression of Genes:

The study permits comparing the influence of the active principle to that of retinoic acid, and the differential expression of the genes in human epiderma reconstructed by the method of “cDNA arrays”.

Epiderma are treated with retinoic acid at a quantity of 10⁻⁶ M or with the active principle according to the invention at a quantity of 2%.

There are then studied the effects of each of the compounds on the expression of selected genes for their role relative to the cells and of the extra-cellular matrix, of the cytokeratins or as to certain proteins of cosmetic interest.

The method used permits noting that the genes discovered as being modulated by retinoic acid are also modulated by the active principle according to the invention, particularly at 2%.

The active principle from alfalfa seeds induces the expression of a gene profile very similar to that of retinoic acid.

### 4/Cosmetic Efficacy in Vivo:

### 4-1/Anti-Wrinkle Effect:

The active principle is formulated at 4% in an emulsion. Retinol is formulated at 0.15% in an emulsion.

The 2 formulae are tests and 2 groups of volunteers, against a placebo.

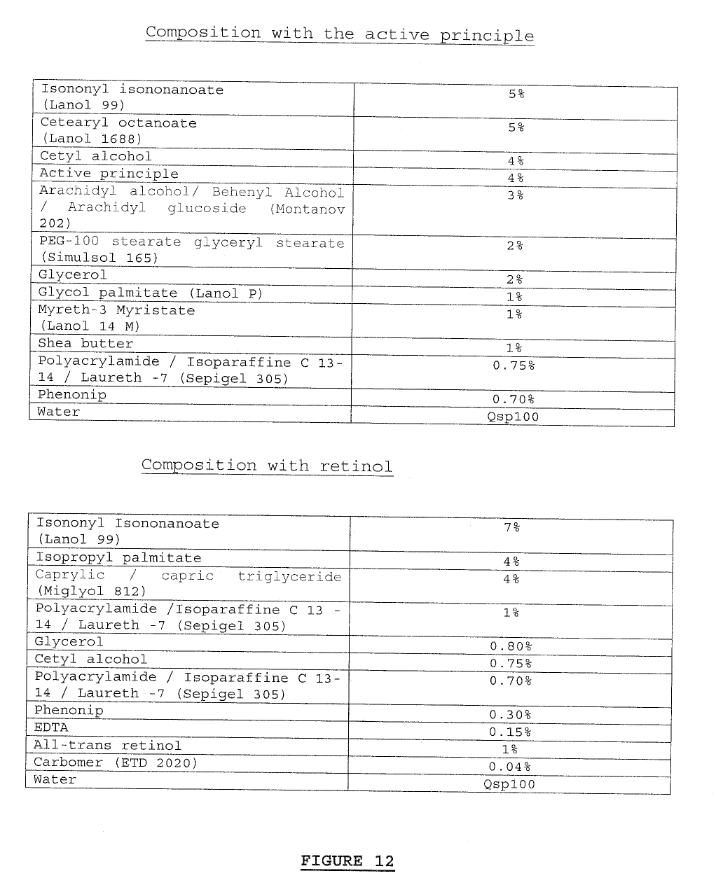

The results obtained are given in the tables of FIG. 12.

The tests have shown the influence of the active principle or of retinol on the appearance of crows-feet wrinkles, after bi-daily application for 28 days.

The analyses were carried out by observation of impressions with the help of a profilometer associated with an image analyzer.

The parameters obtained are:

total wrinkled surface,

total length of wrinkles, and

depth of the wrinkles.

The two compositions applied are mentioned by way of example in the two tables of FIG. 12.

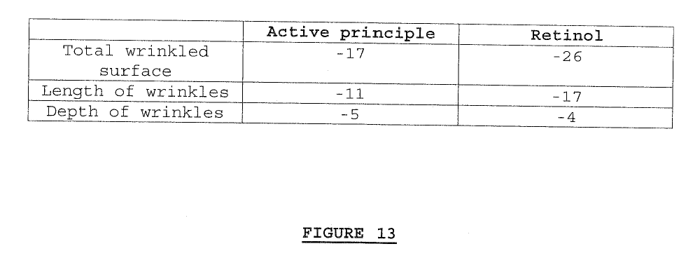

There are obtained the results of the table of FIG. 13.

It will be seen that the active principle has anti-wrinkle properties comparable to those obtained with retinol but without giving rise to irritating reactions.

### 4-2/Smoothing Effect:

The active principle is formulated at 4% in emulsion, and retinol is formulated at 0.15% in an emulsion.

The 2 formulations are tested on 2 groups of volunteers, against a placebo.

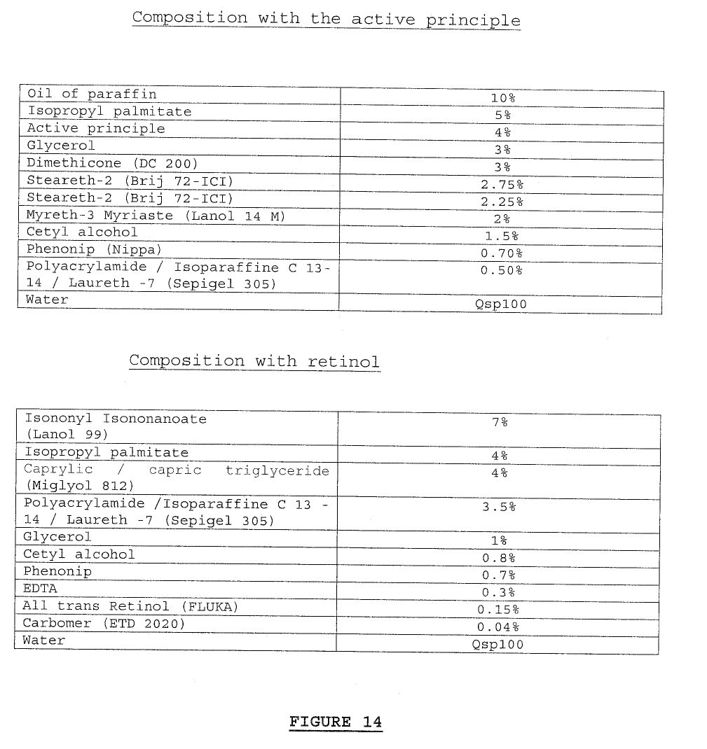

The formulas tested are given in the tables of FIG. 14.

The tests are carried out and the influence of the active principle or of retinol on the appearance of cutaneous micro-relief, after bi-daily application for 28 days.

The analyses are carried out by observation of prints of the cutaneous micro-relief with the help of a profilometer associated with an image analyzer.

The zones in question in this study are cutaneous zones of the forearms. The observed parameter is the mean rugosity of the micro-contour.

The results show a smoothing effect of the active principle on the micro-contour of 8% relative to the comparison, for retinol, and 6% relative to the comparison for the active principle according to the invention.

On the other hand, as indicated at the outset, there are noted reactions of the irritating skin type for volunteers having applied retinol leading to premature exit from the study.

The active principle from alfalfa seeds (Medicago sativa) is particularly active because the different tests and results indicated above show results similar to those obtained with retinol whilst omitting the undesirable effects of retinol.

The invention relates generally to the cosmetic compositions including the active principle according to the invention, in the amount of 0.1 to 20% in a suitable galenic form such as a fatty or aqueous emulsion, a cream, a lotion, an unguent.

1. A method of combating extrinsic aging of the skin to smooth the skin and diminish wrinkles, comprising applying to the skin of a person in need thereof an active principle from alfalfa seed particle (Medicago sativa) in a cosmetalogically acceptable carrier.

2. The method of claim 1, wherein said principle is a fraction rich in galactomannanes.
